(19) United States
(12) Patent Application Publication
(10) Pub. No.: US 2014/0255936 A1
(43) Pub. Date: Sep. 11, 2014

(54) DETECTING FRONTOTEMPORAL DEMENTIA AND AMYOTROPHIC LATERAL SCLEROSIS

(71) Applicants: Rosa Rademakers, Atlantic Beach, FL (US); Mariely DeJesus Hernandez, Jacksonville, FL (US)

(72) Inventors: Rosa Rademakers, Atlantic Beach, FL (US); Mariely DeJesus Hernandez, Jacksonville, FL (US)

(73) Assignee: MAYO FOUNDATION FOR MEDICAL EDUCATION AND RESEARCH, Rochester, MN (US)

(21) Appl. No.: 14/343,807

(22) PCT Filed: Sep. 7, 2012
(86) PCT No.: PCT/US12/54259

§ 371 Date: May 28, 2014

Related U.S. Application Data
(60) Provisional application No. 61/533,125, filed on Sep. 9, 2011.
(60) Provisional application No. 61/534,008, filed on Sep. 13, 2011.

(51) Int. Cl. C12Q 1/68

(52) U.S. Cl. CPC C12Q 1/6883
USPC 435/6.11; 536/24.31; 435/287.2

(57) ABSTRACT

This document provides methods and materials for detecting a nucleic acid expansion. For example, methods and materials for detecting the presence of an expanded number (e.g., greater than 30, 50, 100, 150, 200, 250, 300, 350, 400, 450, 500, 550, 600, 650, 700, or more copies) of a hexanucleotide repeat (e.g., GGGGCC) in the non-coding region of a C9ORF72 gene are provided.

### CROSS-REFERENCE TO RELATED APPLICATIONS

This application claims priority to U.S. Provisional Application Ser. No. 61/534,008, filed on Sep. 13, 2011, and U.S. Provisional Application Ser. No. 61/533,125, filed on Sep. 9, 2011. The disclosures of the prior applications are considered part of (and are incorporated by reference in) the disclosure of this application.

### STATEMENT AS TO FEDERALLY SPONSORED RESEARCH

This invention was made with government support under grants NS065782, AG016574, AG006786, and AG026251 awarded by National Institutes of Health. The government has certain rights in the invention.

### BACKGROUND

1. Technical Field

This document relates to methods and materials related to detecting mammals having frontotemporal dementia (FTD) or amyotrophic lateral sclerosis (ALS). For example, this document relates to methods and materials for using the presence of an expansion of a non-coding GGGGCC hexanucleotide repeat in the gene C9ORF72 to indicate that a mammal has FTD, ALS, or both FTD and ALS.

2. Background Information

FTD and ALS are both devastating neurological diseases. FTD is the second most common cause of pre-senile dementia in which degeneration of the frontal and temporal lobes of the brain results in progressive changes in personality, behavior, and language with relative preservation of perception and memory (Graff-Radford and Woodruff, Neurol., 27:48-57 (2007)). ALS affects 2 in 100,000 people and has traditionally been considered a disorder in which degeneration of upper and lower motor neurons gives rise to progressive spasticity, muscle wasting, and weakness. However, ALS is increasingly recognized to be a multisystem disorder with impairment of frontotemporal functions such as cognition and behavior in up to 50% of patients (Giordana et al., Neurol. Sci., 32:9-16 (2011); Lomen-Hoerth et al., Neurology, 59:1077-1079 (2003); and Phukan et al., Lancet Neurol., 6:994-1003 (2007)). Similarly, as many as half of FTD patients develop clinical symptoms of motor neuron dysfunction (Lomen-Hoerth et al., Neurology, 60:1094-1097 (2002)). The concept that FTD and ALS represent a clinicopathological spectrum of disease is strongly supported by the recent discovery of the transactive response DNA binding protein with a molecular weight of 43 kD (TDP-43) as the pathological protein in the vast majority of ALS cases and in the most common pathological subtype of FTD (Neumann et al., Science, 314:130-133 (2006)), now referred to as frontotemporal lobar degeneration with TDP-43 pathology (FTLD-TDP; Mackenzie et al., Acta Neuropathol., 117:15-18 (2009)).

### SUMMARY

This document provides methods and materials for detecting a nucleic acid expansion. For example, this document provides methods and materials for detecting the presence of an expanded number (e.g., greater than 30, 50, 100, 150, 200, 250, 300, 350, 400, 450, 500, 550, 600, 650, 700, or more copies) of a hexanucleotide repeat (e.g., GGGGCC) in the non-coding region of a C9ORF72 gene. As described herein, a mammal having an expanded number (e.g., greater than 30, 50, 100, 150, 200, 250, 300, 350, 400, 450, 500, 550, 600, 650, 700, or more copies) of GGGGCC repeats within the non-coding region of a C9ORF72 gene can be diagnosed or classified as having FTD, ALS, or both FTD and ALS. In some cases, a mammal having an expanded number of GGGGCC repeats within the non-coding region of a C9ORF72 gene can be diagnosed or classified as having FTD, ALS, or both FTD and ALS as opposed to other forms of dementia such as Alzheimer's disease.

In general, one aspect of this document features a method for diagnosing frontotemporal dementia or amyotrophic lateral sclerosis. The method comprises, or consists essentially of, (a) detecting the presence of an expanded number of GGGGCC repeats located in a C9ORF72 nucleic acid of a human, and (b) classifying the human as having frontotemporal dementia or amyotrophic lateral sclerosis based at least in part on the detection of the presence. The GGGGCC repeats can be located in a non-coding region of the C9ORF72 nucleic acid. The method can comprise detecting the presence of greater than 100 GGGGCC repeats. The method can comprise detecting the presence of greater than 500 GGGGCC repeats. The detecting step can comprise performing a polymerase chain reaction assay. The detecting step can comprise performing a Southern blot assay.

In another aspect, this document features an isolated nucleic acid comprising, or consisting essentially of, a C9ORF72 nucleic acid sequence having greater than 50 GGGGCC repeats. The isolated nucleic acid can have a length between about 350 and about 5,000 bases (e.g., between about 350 and about 4,000 bases, between about 350 and about 3,000 bases, between about 350 and about 2,000 bases, between about 350 and about 1,000 bases, between about 350 and about 750 bases, between about 350 and about 500 bases, or between about 400 and about 1000 bases).

In another aspect, this document features an isolated nucleic acid comprising a C9ORF72 nucleic acid sequence having greater than 100 GGGGCC repeats. The isolated nucleic acid can have a length between about 625 and about 5,000 bases (e.g., between about 625 and about 4,000 bases, between about 625 and about 3,000 bases, between about 625 and about 2,000 bases, between about 625 and about 1,000 bases, between about 625 and about 750 bases, between about 700 and about 2000 bases, or between about 700 and about 1000 bases).

In another aspect, this document features an isolated nucleic acid molecule for performing a Southern blot analysis. The isolated nucleic acid molecule can comprise, or consist essentially of, a C9ORF72 nucleic acid sequence having greater than 20 GGGGCC repeats. The isolated nucleic acid molecule can have a length between about 150 and about 5,000 bases (e.g., between about 150 and about 4,000 bases, between about 150 and about 3,000 bases, between about 150 and about 2,000 bases, between about 150 and about 1,000 bases, between about 150 and about 750 bases, between about 200 and about 2000 bases, or between about 200 and about 1000 bases).

In another aspect, this document features a container comprising, or consisting essentially of, a population of isolated nucleic acid molecules. The isolated nucleic acid molecules comprise, or consist essentially of, a C9ORF72 nucleic acid sequence having greater than 10 GGGGCC repeats, wherein the population comprises at least five different isolated nucleic acid molecules each with a different number of GGGGCC repeats. The isolated nucleic acid molecule can have a length between about 65 and about 5,000 bases (e.g., between about 65 and about 4,000 bases, between about 65 and about 3,000 bases, between about 65 and about 2,000 bases, between about 65 and about 1,000 bases, between about 65 and about 750 bases, between about 65 and about 2000 bases, or between about 65 and about 1000 bases). The isolated nucleic acid molecules can comprise a fluorescent label (e.g., a FAM label).

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 contains results demonstrating that an expanded GGGGCC hexanucleotide repeat in C9ORF72 causes FTD and ALS linked to chromosome 9p in family VSM-20. Panel A is a graph plotting the segregation of GGGGCC repeat in C9ORF72 and flanking genetic markers in disguised linkage pedigree of family VSM-20. The arrowhead denotes the proband. For the GGGGCC repeat, numbers indicate hexanucleotide repeat units, and the X denotes that the allele could not be detected. Black symbols represent patients affected with frontotemporal dementia (left side filled), amyotrophic lateral sclerosis (right side filled), or both. White symbols represent unaffected individuals or at-risk individuals with unknown phenotype. Haplotypes for individuals 20-1, 20-2, and 20-3 are inferred from genotype data of siblings and offspring. Panel B contains graphs plotting the fluorescent fragment length analyses of a PCR fragment containing the GGGGCC repeat in C9ORF72 for the indicated members of family VSM-20. PCR products from the unaffected father (20-9), affected mother (2-10), and their offspring (20-16, 20-17, and 20-18) are shown illustrating the lack of transmission from the affected parent to affected offspring. Numbers under the peaks indicate number of GGGGCC hexanucleotide repeats. Panel C contains graphs plotting the PCR products of repeat-primed PCR reactions separated on an ABI3730 DNA Analyzer and visualized by GENEMAPPER software for the indicated members of family VSM-20. Electropherograms are zoomed to 2000 relative fluorescence units to show stutter amplification. Two expanded repeat carriers (20-8 and 20-15) and one non-carrier (20-5) from family VSM-20 are shown. Panel D is a photograph of a Southern blot of four expanded repeat carriers and one non-carrier from family member of VSM-20 using genomic DNA extracted from lymphoblast cell lines. Lane 1 shows DIG-labeled DNA Molecular Weight Marker II (Roche) with fragments of 2027, 2322, 4361, 6557, 9416, 23130 bp, lane 2 shows DIG-labeled DNA Molecular Weight Marker VII (Roche) with fragments of 1882, 1953, 2799, 3639, 4899, 6106, 7427, and 8576 bp. Patients with expanded repeats (lanes 3-6) show an additional allele from 6-12 kb, while a normal relative (lane 7) only shows the expected 2.3 kb wild-type allele.

FIG. 2 is a graph demonstrating a correlation of GGGGCC hexanucleotide repeat length with rs3849942, a surrogate marker for the previously published chromosome 9p ‘risk’ haplotype. The histogram presents the number of GGGGCC repeats in 505 controls homozygous for the rs3849942 G-allele (GG) and in 49 controls homozygous for the rs3849942 A-allele (AA).

FIG. 3 contains results demonstrating the effect of expanded hexanucleotide repeat on C9ORF72 expression. Panel A is a diagram of an overview of the genomic structure of the C9ORF72 locus (top portion) and the C9ORF72 transcripts produced by alternative pre-mRNA splicing (bottom portion). Boxes represent coding (white) and non-coding (grey) exons, and the positions of the start codon (ATG) and stop codon (TAA) are indicated. The GGGGCC repeat is indicated with a diamond. The position of rs10757668 is indicated with a star. Panel B contains sequence traces of C9ORF72 exon 2 spanning rs10757668 in gDNA (top trace) and cDNA (bottom traces) prepared from frontal cortex of an FTLD-TDP patient carrying an expanded GGGGCC repeat. The arrow indicates the presence of the wild-type (G) and mutant (A) alleles of rs10757668 in gDNA. Transcript specific cDNAs were amplified using primers spanning the exon 1b/exon 2 boundary (variant 1) or exon 1a/exon 2 boundary (variant 2 and 3). Sequenced traces derived from cDNA transcripts indicate the loss of variant 1 but not variant 2 or 3 mutant RNA. Similar results were obtained for two unrelated FTLD-TDP mutation carriers. The bottom trace shows a non-expanded repeat carrier heterozygous for rs10757668 to confirm the presence of both alleles of transcript variant 1 validating the method. Panel C contains graphs plotting results from an mRNA expression analysis of C9ORF72 transcript variant 1 using a custom-designed Taqman expression assay. Top graph shows results from lymphoblast cell lines derived from expanded repeat carriers from family VSM-20 (n=7) and controls (n=7), and the bottom graph shows results from RNA extracted from frontal cortex brain samples from FTLD-TDP patients with (n=7) and without (n=7) the GGGGCC repeat expansion. Data indicate mean±s.e.m. ** indicates P<0.01. Panel D contain graphs plotting results from an mRNA expression analysis of all C9ORF72 transcripts encoding for C9ORF72 isoform a (variant 1 and 3) using inventoried ABI Taqman expression assay Hs_{—}00945132. The top graph shows results using RNA extracted from lymphoblast cell lines derived from expanded repeat carriers from family VSM-20 (n=7) and controls (n=7), and the bottom graph shows results using RNA extracted from frontal cortex brain samples from FTLD-TDP patients with (n=7) and without (n=7) the GGGGCC repeat expansion. Data indicate mean±s.e.m. * indicates P<0.05.

FIG. 4 contains results demonstrating that expanded GGGGCC hexanucleotide repeats form nuclear RNA foci in human brain and spinal cord. Panel A is a photograph of multiple RNA foci in the nucleus (stained with DAPI, blue) of a frontal cortex neuron of the proband of family 63 (63-1) using a Cy3-labeled (GGCCCC)₄ oligonucleotide probe (red label). Multiple red foci were observed. Panel B is a photograph of RNA foci observed in the nucleus of two lower motor neurons in FTD/ALS patient (13-7) carrying an expanded GGGGCC repeat using a Cy3-labeled (GGCCCC)₄ oligonucleotide probe. Multiple red foci were observed within each nucleus. Panel C is a photograph of the absence of RNA foci in the nucleus of cortical neuron from FTLD-TDP patient (44-1) without an expanded GGGGCC repeat in C9ORF72. Panel D is a photograph of spinal cord tissue sections from patient 13-7 probed with a Cy3-labeled (CAGG)₆ oligonucleotide probe (negative control probe). Spinal cord tissue sections from patient 13-7 exhibited RNA foci with the (GGCCCC)₄ oligonucleotide probe (panel B), but did not show any foci with a Cy3-labeled (CAGG)₆ oligonucleotide probe (negative control probe) (Panel D). Scale bar: 10 μm (A and C), 20 μm (B and D).

FIG. 5 contains photographs of the neuropathology in familial FTD/ALS linked to chromosome 9p (family VSM-20). Panels A and B are photographs of FTLD-TDP tissue characterized by TDP-43 immunoreactive neuronal cytoplasmic inclusions and neurites in (A) neocortex and (B) hippocampal dentate granule cell layer. Panel C is a photograph of TDP-34 immunoreactive neuronal cytoplasmic inclusions in spinal cord lower motor neurons, typical of ALS. Panel D is a photograph of numerous neuronal cytoplasmic inclusions and neurites in cerebellar granular layer immunoreactive for ubiquitin but not TDP-43. Scale bar: (A) 15 μm, (B) 30 μm, (C) 100 μm, (D) 12 μm.

FIG. 6 contains results from additional families with an expanded hexanucleotide repeat in C9ORF72. Panel A is a graph of abbreviated pedigrees of families with expanded repeats for which DNA samples of multiple affected individuals were available. Probands from families 2, 13, 32, and 63 were part of the UBC FTLD-TDP cohort, while probands of families 118, 125, and 158 were ascertained at MCR and part of the MC Clinical FTD series. Black symbols represent patients affected with frontotemporal dementia (left side filled), amyotrophic lateral sclerosis (right side filled), or both. Grey symbols represent individuals affected with an unspecified neurodegenerative disorder. White symbols represent unaffected individuals or at-risk individuals with unknown phenotype. To protect confidentiality, some individuals are not shown, and sex is portrayed using a diamond for all individuals except for affected individuals and their spouse. Autopsy confirmation of FTLD-TDP is indicated with a pound sign (#). A ‘+’ sign indicates that DNA was included in the genetic analyses to confirm that mutations segregated with disease. Panel B is a photograph of representative Southern blots of DNA extracted from peripheral blood (lanes 1-6), brain (lane 7), and lymphoblast cells (lane 8) of patients with and without expanded repeats in C9ORF72 selected from an FTD and ALS patient series. Expanded repeat carriers are indicated with ‘X’, non-carriers are indicated with ‘N. Note the smear of high molecular weight bands in DNA extracted from blood and brain suggesting somatic instability of the repeat.

FIG. 7 contains results demonstrating the characterization of C9ORF72 mRNA transcripts and C9ORF72 immunohistochemistry in normal and affected brain tissue. Panel A is a photograph of an agarose gel-electrophoresis of RT-PCR products generated from normal frontal cortex brain using primers designed to known C9ORF72 transcript variants 1 (V1, NM_{—}145005.4) and 2 (V2, NM_{—}018325.2). The V1 lane shows the expected 442 bp size band. The V2 lane shows the expected band at 484 bp and an unexpected larger band (arrow). Sequence analysis of this product determined an additional alternative spliced C9ORF72 transcript (variant 3, V3) resulting from the fact that exon 1a reads through the donor site and is lengthened by 78 bp of intronic sequence. RT-PCR analysis revealed that transcript V3 extends full length to exon 11 and is therefore predicted to encode for C9ORF72 isoform a similar to V1. Panel B contains sequence traces using isoform specific primers. The differing sequence chromatograms of the exon 1/exon 2 boundary in the three transcripts of C9ORF72 are shown. Panel C contains photographs of an RT-PCR analysis of C9ORF72 using a forward primer specific to each of the three transcripts and a reverse primer located in C9ORF72 exon 2. Expression of all three isoforms was observed in a range of normal human tissues, including multiple brain regions. High quality RNA from kidney, liver, lung, heart, testis, and fetal brain tissues were purchased from Cell Applications, while RNA from the adult human brain regions was extracted from normal brain samples selected from the MCF brain bank. Lymphoblast RNA was extracted from a normal healthy control individual. Panel D is a photograph of immunoblotting of C9ORF72 in lymphoblast cell line lysates from GGGGCC repeat carriers (+) and non-carriers (−). Cell lysate extracted from HeLa was included in the last lane as a positive control (denoted by C) to verify molecular weight of the C9ORF72 protein. A GAPDH antibody was used as a protein loading control. Panel E is a photograph of immunoblotting of C9ORF72 in frontal cortex lysates from FTLD-TDP patients with expanded repeats (+) and FTLD-TDP patients without expanded repeats (−). Brains with normal repeat length free of TDP-43 pathology were also included. A GAPDH antibody was used as a protein loading control. Panels F-H are photographs of C9ORF72 immunohistochemistry in patients with GGGGCC repeat expansion. In cases of ALS with and without the repeat expansion, some lower motor neurons that appeared to be chromatolytic showed more intense diffuse cytoplasmic reactivity, but there was no staining of inclusion bodies (spinal cord lower motor neurons (Panel F)). Swollen axons (arrows) in ventral spinal cord showed intense immunoreactivity; however, these were also present in many cases of ALS without C9ORF72 repeat expansion (Panel G). Hippocampal pyramidal neurons were surrounded by coarse punctate staining, consistent with large presynaptic terminals (Panel H). This pattern was more prominent in cases of FTLD compared with normal controls, but was not specific for cases with C9ORF72 repeat expansion. Scale bar: (F, G) 40 μm, (H) 20 μm.

FIG. 8 is a listing of C9ORF72 nucleic acid upstream and downstream of the GGGGCC repeat expansion site (SEQ ID NO:1). The GGGGCC repeat expansion site is in bold and underlined.

FIG. 9 is a Southern blot analysis of GGGGCC repeat expansions using DNA extracted from several brain regions, peripheral tissues, and blood from a patient diagnosed with progressive muscular atrophy (PMA) without upper motor neuron signs. Lane 1, spleen; lane 2, spleen; lane 3, heart; lane 4, muscle; lane 5, blood; lane 6, liver; lane 7, frontal cortex; lane 8, temporal cortex; lane 9, cerebellum; and lane 10, positive control cell line.

### DETAILED DESCRIPTION

This document provides methods and materials related to detecting a nucleic acid expansion. For example, this document provides methods and materials for detecting the presence of an expanded number (e.g., greater than 30, 50, 100, 150, 200, 250, 300, 350, 400, 450, 500, 550, 600, 650, 700, or more copies) of a hexanucleotide repeat (e.g., GGGGCC) in a C9ORF72 gene (e.g., in the non-coding region of a C9ORF72 gene). As described herein, a mammal having an expanded number (e.g., greater than 30, 50, 100, 150, 200, 250, 300, 350, 400, 450, 500, 550, 600, 650, 700, or more copies) of GGGGCC repeats within a C9ORF72 gene (e.g., within a non-coding region of a C9ORF72 gene) can be diagnosed or classified as having FTD, ALS, or both FTD and ALS. In some cases, a mammal having an expanded number of GGGGCC repeats within a C9ORF72 gene (e.g., within a non-coding region of a C9ORF72 gene) can be diagnosed or classified as having FTD, ALS, or both FTD and ALS as opposed to other forms of dementia or neurological conditions such as Alzheimer's disease, Parkinson's disease, dementia with lewy bodies (LBD), corticobasal syndrome, or progressive supranuclear palsy.

The mammal can be any type of mammal including, without limitation, a dog, cat, horse, sheep, goat, cow, pig, monkey, or human. The methods and materials provided herein can be used to determine whether or not a mammal (e.g., human) contains nucleic acid having the presence of an expanded number (e.g., greater than 30, 50, 100, 150, 200, 250, 300, 350, 400, 450, 500, 550, 600, 650, 700, or more copies) of a hexanucleotide repeat (e.g., GGGGCC) in a C9ORF72 gene (e.g., in a non-coding region of a C9ORF72 gene). In some cases, the methods and materials provided herein can be used to determine whether one or both alleles containing a C9ORF72 gene contain the presence of an expanded number (e.g., greater than 30, 50, 100, 150, 200, 250, 300, 350, 400, 450, 500, 550, 600, 650, 700, or more copies) of a hexanucleotide repeat (e.g., GGGGCC) in a C9ORF72 gene (e.g., in a non-coding region of a C9ORF72 gene). The identification of the presence of an expanded number of a hexanucleotide repeat (e.g., GGGGCC) in a C9ORF72 gene (e.g., in a non-coding region of a C9ORF72 gene) can be used to diagnose FTD, ALS, or both FTD and ALS in a mammal, typically when known clinical symptoms of a neurological disorder also are present or when the mammal is “at risk” to develop the disease, e.g., because of a family history of an expanded number of hexanucleotide repeats in C9ORF72. In some cases, a mammal (e.g., a human) having an expanded number of a hexanucleotide repeat (e.g., GGGGCC) in a C9ORF72 gene (e.g., in a non-coding region of a C9ORF72 gene) can be diagnosed as having FTD, ALS, or both FTD and ALS independent of whether that mammal already exhibits symptoms or someone in their family already has symptoms.

As described herein, a human who (a) is experiencing clinical symptoms of a neurological disorder or has a family history of a neurological disorder (e.g., FTD or ALS) and (b) has greater than 30 copies of a GGGGCC repeat within in a C9ORF72 gene can be classified or diagnosed as having FTD, ALS, or both FTD and ALS. For example, a son whose mother is known to have had FTD and ALS can be classified as having FTD and ALS if it is determined that the son contains greater than 30 copies (e.g., greater than 50, 100, 150, 200, 250, 300, 350, 400, 450, 500, 550, 600, 650, 700, or more copies) of a GGGGCC repeat within in a C9ORF72 gene.

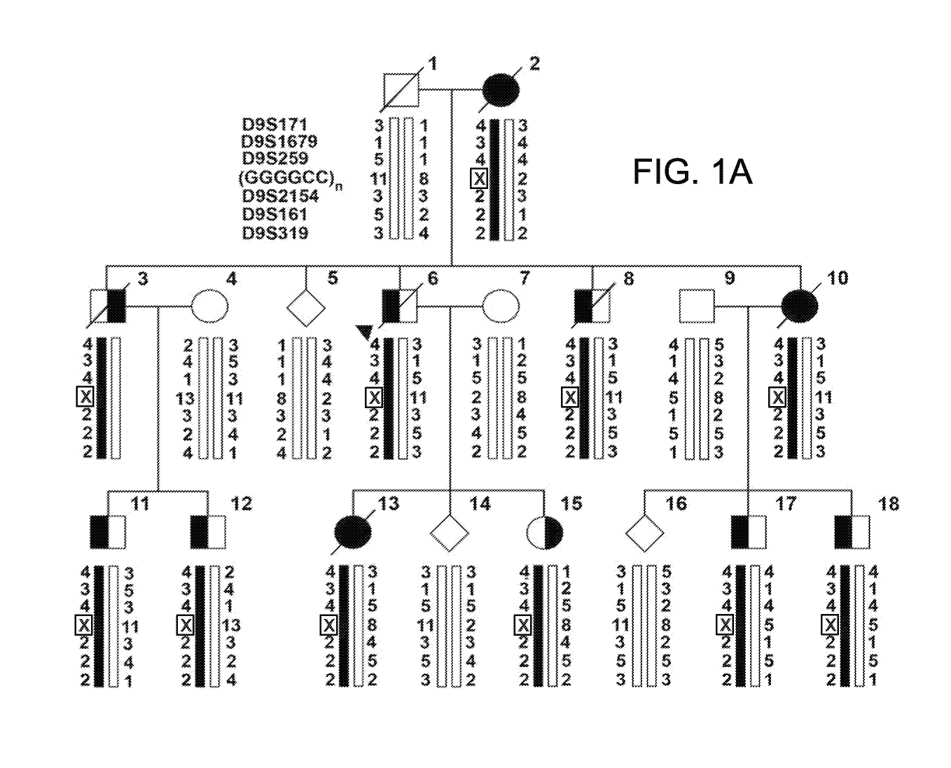

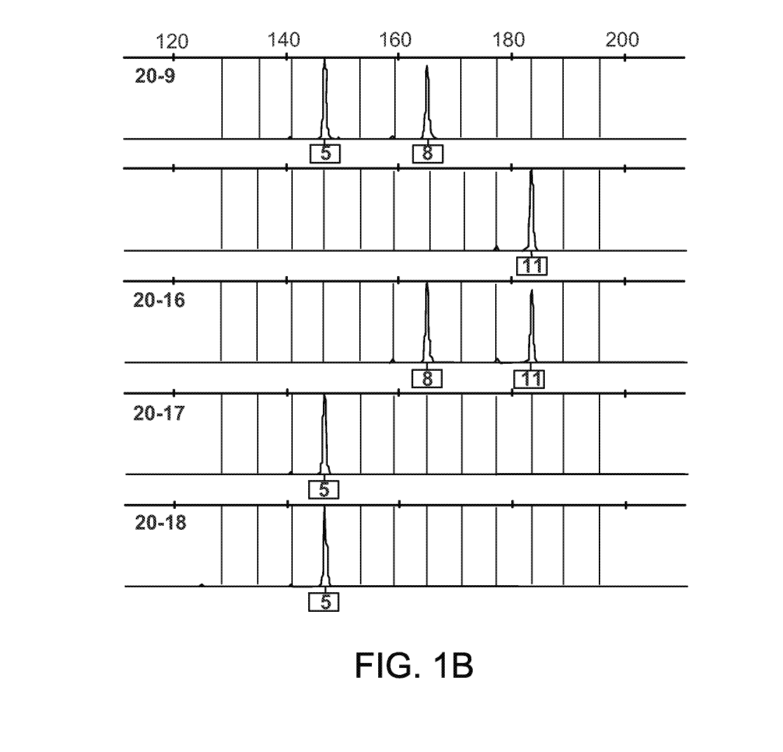

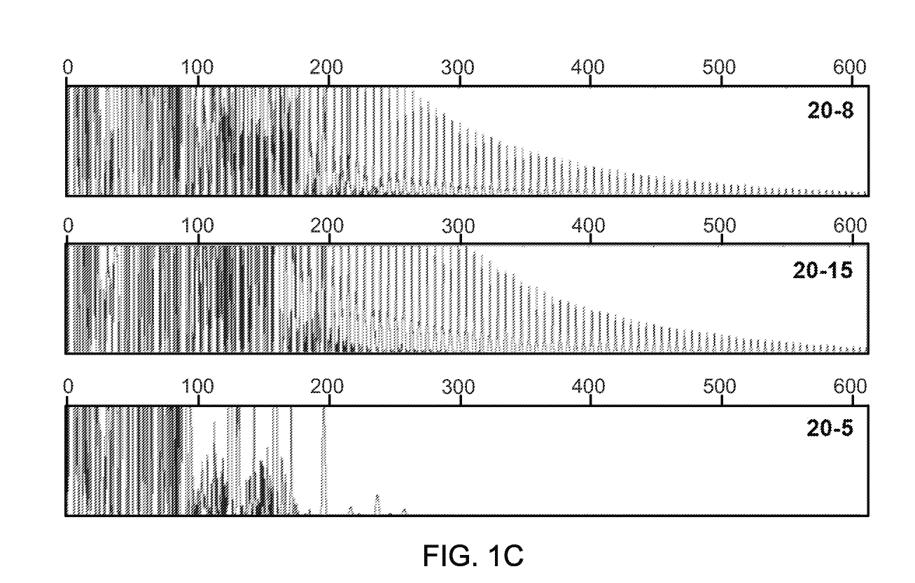

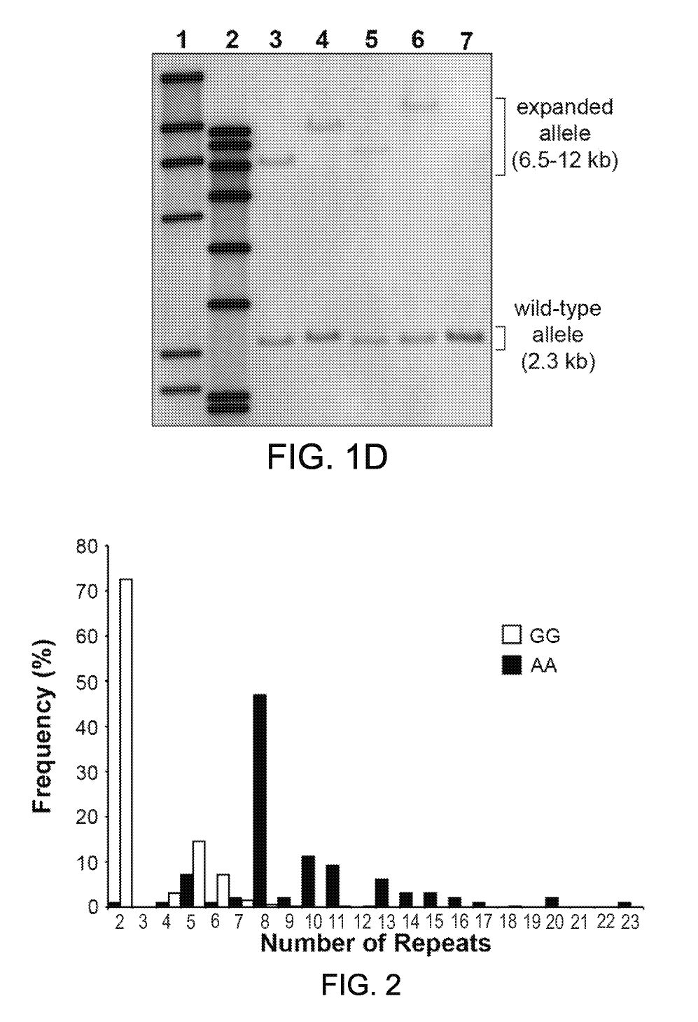

Any appropriate method can be used to detect the presence of an expanded number of a hexanucleotide repeat (e.g., GGGGCC) in a C9ORF72 gene (e.g., in a non-coding region of a C9ORF72 gene). For example, PCR-based assays such as those described herein can be used to detect the presence of an expanded number of a hexanucleotide repeat (e.g., GGGGCC) in the non-coding region of a C9ORF72 gene. Briefly, a labeled primer (e.g., MRX-F primer) designed to hybridize upstream of the GGGGCC site of a C9ORF72 gene can be used in an amplification reaction in combination with a primer designed to hybridize within the GGGGCC repeat (e.g., MRX-R1). Any appropriate label can be used including, without limitation, Cy5, Cy3, or 6-carboxyfluorescein. The primer designed to hybridize within the GGGGCC repeat can include a tail sequence (e.g., M13 sequence) that can serve as a template for a third primer (e.g., MRX-M13R). Any appropriate sequence can be used as the tail sequence and the third primer provided that they are capable of hybridizing to each other. Analysis of the results from an amplification reaction using these three primers can indicate whether a sample (e.g., genomic DNA sample) contains an allele having an expanded number of GGGGCC repeats in a C9ORF72 gene. Examples of such results are provided in FIG. 1C.

In some cases, Southern blotting techniques can be used to detect the presence of an expanded number of a hexanucleotide repeat (e.g., GGGGCC) in a C9ORF72 gene (e.g., in a non-coding region of a C9ORF72 gene). For example, a patient's nucleic acid can be assessed using a probe designed to hybridize to a region that includes at least a portion of the GGGGCC site of a C9ORF72 gene. In some cases, a Southern blotting technique can be used to determine the number of GGGGCC repeats in a C9ORF72 gene in addition to detecting the presence or absence of an expanded number of GGGGCC repeats.

In some cases, genomic DNA can be used to detect the presence of an expanded number of a hexanucleotide repeat (e.g., GGGGCC) in a C9ORF72 gene (e.g., in a non-coding region of a C9ORF72 gene). Genomic DNA typically is extracted from a biological sample such as a peripheral blood sample, but can be extracted from other biological samples, including tissues (e.g., mucosal scrapings of the lining of the mouth or from renal or hepatic tissue). Any appropriate method can be used to extract genomic DNA from a blood or tissue sample, including, for example, phenol extraction. In some cases, genomic DNA can be extracted with kits such as the QIAamp® Tissue Kit (Qiagen, Chatsworth, Calif.), the Wizard® Genomic DNA purification kit (Promega, Madison, Wis.), the Puregene DNA Isolation System (Gentra Systems, Minneapolis, Minn.), or the A.S.A.P.3 Genomic DNA isolation kit (Boehringer Mannheim, Indianapolis, Ind.).

As described herein, the presence of an expanded number of a hexanucleotide repeat (e.g., GGGGCC) in a C9ORF72 gene (e.g., in a non-coding region of a C9ORF72 gene) in a mammal (e.g., human) can indicate that that mammal has FTD, ALS, or both FTD and ALS. In some cases, the presence of an expanded number of a hexanucleotide repeat (e.g., GGGGCC) in a C9ORF72 gene (e.g., in a non-coding region of a C9ORF72 gene) in a human can indicate that that human has FTD, ALS, or both FTD and ALS, especially when that human is between the ages of 30 and 80, has a family history of dementia, and/or presents symptoms of dementia. Symptoms of dementia can include changes in behavior such as changes that result in impulsive, repetitive, compulsive, or even criminal behavior. For example, changes in dietary habits and personal hygiene can be symptoms of dementia. Symptoms of dementia also can include language dysfunction, which can present as problems in expression of language, such as problems using the correct words, naming objects, or expressing one's self. Difficulties reading and writing can also develop. In some cases, the presence of an expanded number of a hexanucleotide repeat (e.g., GGGGCC) in a C9ORF72 gene (e.g., in a non-coding region of a C9ORF72 gene), together with positive results of other diagnostic tests, can indicate that the mammal has FTD, ALS, or both FTD and ALS. For example, the presence of an expanded number of a hexanucleotide repeat (e.g., GGGGCC) in the non-coding region of a C9ORF72 gene together with results from a neurological exam, neurophysical testing, cognitive testing, and/or brain imaging can indicate that a mammal has FTD, ALS, or both FTD and ALS.

In some cases, the methods and materials provided herein can be used to assess human patients for inclusion in or exclusion from a treatment regimen or a clinical trial. For example, patients identified as having FTD, ALS, or both FTD and ALS, as opposed to Alzheimer's disease, using the methods and materials provided herein can be removed from a treatment regimen designed to treat Alzheimer's disease. In another example, patients being considered for inclusion in a clinical study for Alzheimer's disease can be excluded based on the presence of an expanded number of a hexanucleotide repeat (e.g., GGGGCC) in a C9ORF72 gene as described herein.

This document also provides methods and materials for treating patients having FTD, ALS, or both FTD and ALS. For example, a patient suspected of having FTD, ALS, or both FTD and ALS based on, for example, a family history of dementia and/or symptoms of dementia, can be assessed for the presence of an expanded number of a hexanucleotide repeat (e.g., GGGGCC) in a C9ORF72 gene (e.g., in a non-coding region of a C9ORF72 gene) to identify that patient as having FTD, ALS, or both FTD and ALS. Once identified as having FTD, ALS, or both FTD and ALS based at least in part on the presence of an expanded number of a hexanucleotide repeat (e.g., GGGGCC) in a C9ORF72 gene (e.g., in a non-coding region of a C9ORF72 gene), the patient can be administered or instructed to self-administer one or more agents designed to reduce the symptoms or progression of FTD or ALS. An example of an agent designed to reduce the progression of FTD is riluzole.

This document also provides nucleic acid molecules that include at least a portion of a C9ORF72 nucleic acid sequence and an expanded number (e.g., greater than 30, 50, 100, 150, 200, 250, 300, 350, 400, 450, 500, 550, 600, 650, 700, or more copies) of a hexanucleotide repeat (e.g., GGGGCC). The term “nucleic acid” as used herein encompasses both RNA and DNA, including cDNA, genomic DNA, and synthetic (e.g., chemically synthesized) DNA. A nucleic acid can be double-stranded or single-stranded. A single-stranded nucleic acid can be the sense strand or the antisense strand. In addition, a nucleic acid can be circular or linear.

An “isolated nucleic acid” refers to a nucleic acid that is separated from other nucleic acid molecules that are present in a naturally-occurring genome, including nucleic acids that normally flank one or both sides of the nucleic acid in a naturally-occurring genome. The term “isolated” as used herein with respect to nucleic acids also includes any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., any paramyxovirus, retrovirus, lentivirus, adenovirus, or herpes virus), or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not considered an isolated nucleic acid.

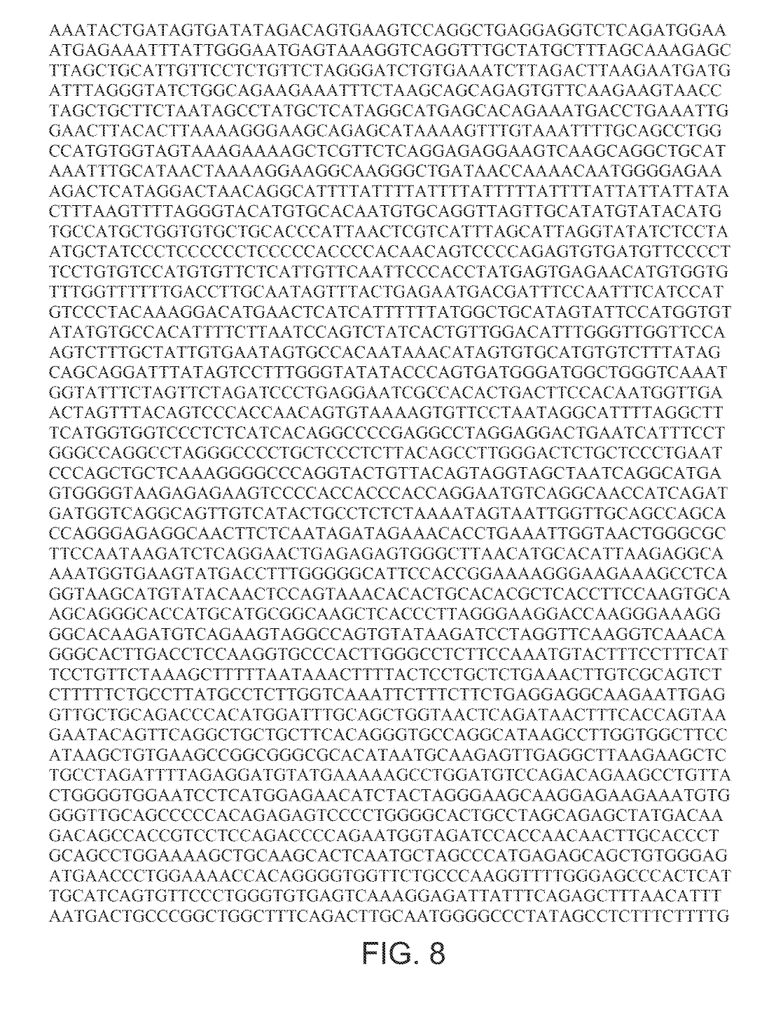

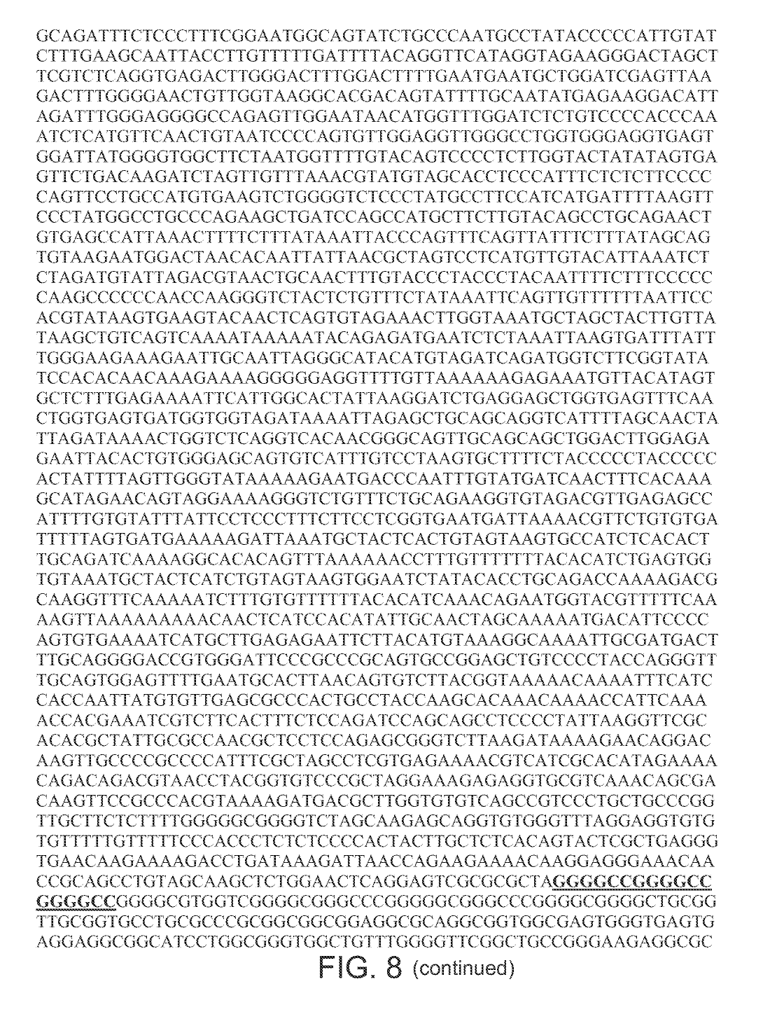

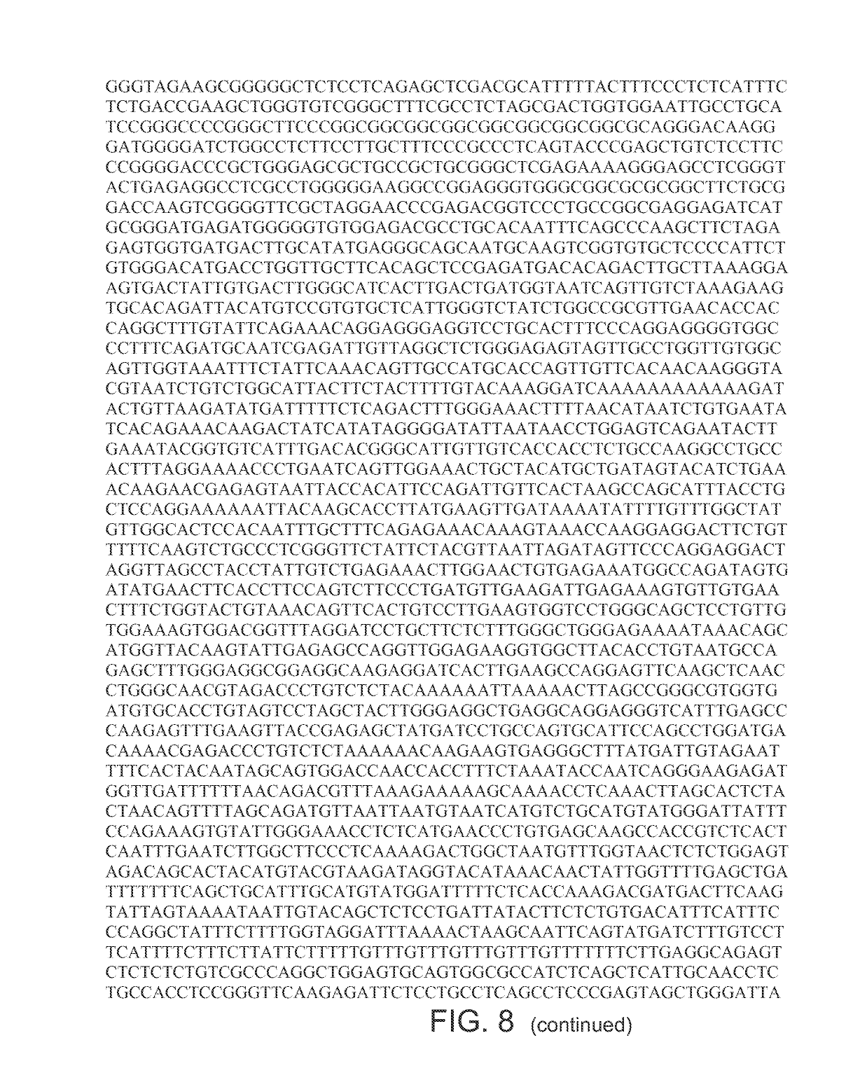

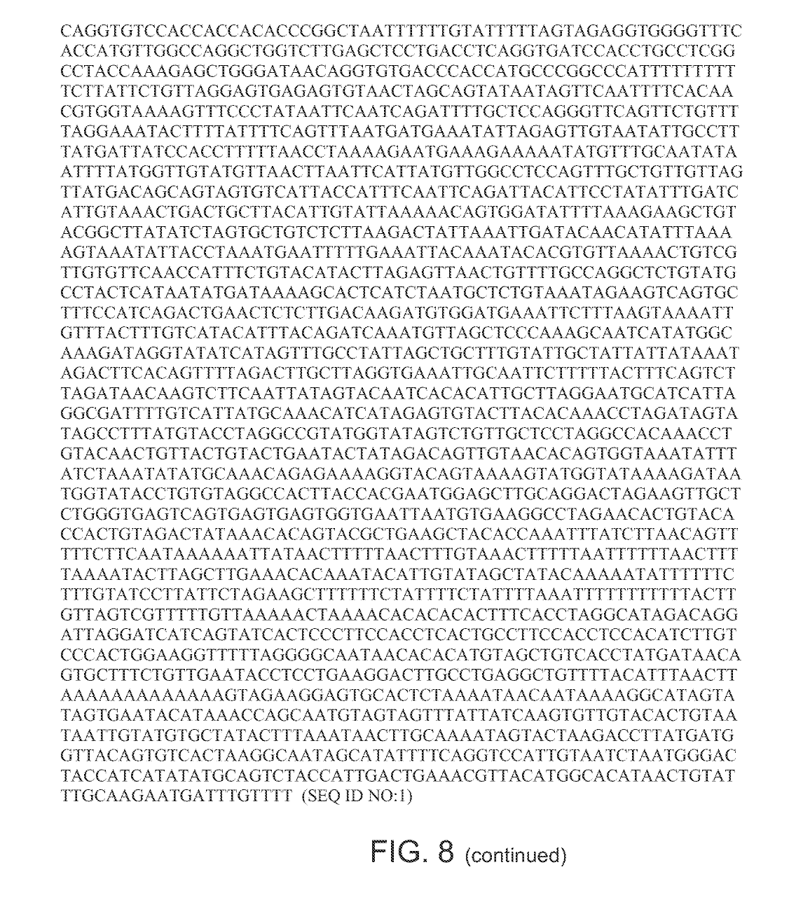

An isolated nucleic acid provided herein can include at least a portion of a C9ORF72 nucleic acid sequence (e.g., a non-coding C9ORF72 nucleic acid sequence) and an expanded number (e.g., greater than 30, 50, 100, 150, 200, 250, 300, 350, 400, 450, 500, 550, 600, 650, 700, or more copies) of a hexanucleotide repeat (e.g., GGGGCC). For example, an isolated nucleic acid provided herein can include at least a portion of the C9ORF72 nucleic acid sequence set forth in SEQ ID NO:1 provided that the bold and underlined GGGGCC repeat site contains an expanded number (e.g., greater than 30, 50, 100, 150, 200, 250, 300, 350, 400, 450, 500, 550, 600, 650, 700, or more copies) of GGGGCC units in place of the three GGGGCC units shown in FIG. 8. In some cases, an isolated nucleic acid provided herein can include a C9ORF72 nucleic acid sequence (e.g., a C9ORF72 nucleic acid sequence set forth in SEQ ID NO:1) that is from about 5 to about 5000 nucleotides in length (e.g., from about 5 to about 2500, from about 5 to about 1000, from about 5 to about 500, from about 5 to about 250, from about 5 to about 200, from about 5 to about 150, from about 5 to about 100, from about 10 to about 500, or from about 20 to about 500 nucleotides in length) and that is upstream of a hexanucleotide repeat site (e.g., a GGGGCC site), followed by an expanded number (e.g., greater than 30, 50, 100, 150, 200, 250, 300, 350, 400, 450, 500, 550, 600, 650, 700, or more copies) of a hexanucleotide repeat (e.g., GGGGCC), followed by a C9ORF72 nucleic acid sequence (e.g., a C9ORF72 nucleic acid sequence set forth in SEQ ID NO:1) that is from about 5 to about 5000 nucleotides in length (e.g., from about 5 to about 2500, from about 5 to about 1000, from about 5 to about 500, from about 5 to about 250, from about 5 to about 200, from about 5 to about 150, from about 5 to about 100, from about 10 to about 500, or from about 20 to about 500 nucleotides in length) and that is downstream of that hexanucleotide repeat site (e.g., a GGGGCC site). In some cases, an isolated nucleic acid provided herein can include a C9ORF72 nucleic acid sequence (e.g., a C9ORF72 nucleic acid sequence set forth in SEQ ID NO:1) that is from about 5 to about 5000 nucleotides in length (e.g., from about 5 to about 2500, from about 5 to about 1000, from about 5 to about 500, from about 5 to about 250, from about 5 to about 200, from about 5 to about 150, from about 5 to about 100, from about 10 to about 500, or from about 20 to about 500 nucleotides in length) and that is upstream of a hexanucleotide repeat site (e.g., a GGGGCC site), followed by an expanded number (e.g., greater than 30, 50, 100, 150, 200, 250, 300, 350, 400, 450, 500, 550, 600, 650, 700, or more copies) of a hexanucleotide repeat (e.g., GGGGCC). In some cases, an isolated nucleic acid provided herein can include an expanded number (e.g., greater than 30, 50, 100, 150, 200, 250, 300, 350, 400, 450, 500, 550, 600, 650, 700, or more copies) of a hexanucleotide repeat (e.g., GGGGCC), followed by a C9ORF72 nucleic acid sequence (e.g., a C9ORF72 nucleic acid sequence set forth in SEQ ID NO:1) that is from about 5 to about 5000 nucleotides in length (e.g., from about 5 to about 2500, from about 5 to about 1000, from about 5 to about 500, from about 5 to about 250, from about 5 to about 200, from about 5 to about 150, from about 5 to about 100, from about 10 to about 500, or from about 20 to about 500 nucleotides in length) and that is downstream of that hexanucleotide repeat site (e.g., a GGGGCC site).

The invention will be further described in the following examples, which do not limit the scope of the invention described in the claims.

### EXAMPLES

### Example 1

### Expanded GGGGCC Hexanucleotide Repeat in Non-Coding Region of C9ORF72 Causes Chromosome 9p-Linked Frontotemporal Dementia and Amyotrophic Lateral Sclerosis

### Human Samples

Four extensive FTD and ALS patient cohorts and one control cohort were included in this study. All individuals agreed to be in the study and biological samples were obtained after informed consent from subjects and/or their proxies. Demographic and clinical information for each cohort was summarized in Table 1. The proband of chromosome 9p-linked family VSM-20 was part of a series of 26 probands ascertained at UBC, Vancouver, Canada, characterized by a pathological diagnosis of FTLD with TDP-43 pathology (FTLD-TDP) and a positive family history of FTD and/or ALS (UBC FTLD-TDP cohort). Clinical and pathological evaluations of VSM-20 were conducted at UCSF, UBC and the Mayo Clinic (Boxer et al., J. Neurol. Neurosurg. Psychiatry, 82: 196-203 (2011)). A second cohort of 93 pathologically confirmed FTLD-TDP patients independent of family history was selected from the Mayo Clinic Florida (MCF) brain bank (MCF FTLD-TDP cohort) which focused predominantly on dementia. The clinical FTD cohort (MC Clinical FTD cohort) was ascertained by the Behavioral Neurology sections at MCF (n=197) and MCR (n=177), the majority of whom were participants in the Mayo Alzheimer's Disease Research Center. Members of Family 118 were participants in the Mayo Alzheimer's Disease Patient Registry.

Clinical FTD patients underwent a full neurological evaluation and all who were testable had a neuropsychological evaluation. Structural neuroimaging was performed in all patients and functional imaging was performed in many patients. Patients with a clinical diagnosis of behavioral variant FTD (bvFTD), semantic dementia or progressive non-fluent aphasia based on Neary criteria (Neary et al., Neurology, 51:1546-1554 (1998)) or patients with the combined phenotype of bvFTD and ALS were included in this study, while patients with a diagnosis of logopenic aphasia or corticobasal syndrome were excluded. In the MCF FTLD-TDP cohort and the MC Clinic FTD cohort, a positive family history was defined as a first or second degree relative with FTD and/or ALS or a first degree relative with memory problems, behavioral changes, parkinsonism, schizophrenia, or another suspected neurodegenerative disorder. It should be noted that information about family history was lacking in a significant proportion (23.7%) of the MCF FTLD-TDP cohort and these were included in the “sporadic” group. A cohort of 229 clinical ALS patients was ascertained by the ALS Center at MCF (MCF clinical ALS cohort). These patients underwent a full neurological evaluation including electromyography, clinical laboratory testing and imaging as appropriate to establish the clinical diagnosis of ALS. A positive family history in the MCF ALS series was defined as a first or second degree relative with ALS. The Control cohort (n=909) was comprised of DNA samples from 820 control individuals collected from the Department of Neurology and DNA extracted from 89 normal control brains from the MCF brain bank.

TABLE 1
Demographics of patient and control cohorts analyzed for the presence
of the chromosome 9p GGGGCC repeat expansion in C9ORF72.

| | | | | Positive | |
|---|---|---|---|---|---|
| Study | | Age^{a} | | family | Diagnosis |
| cohorts | N | (years) | Females | History^{b} | (N) |
| UBC | 26 | 61.0 ± 11.4 | 10 (38.5%) | 100% | FTLD-TDP |
| FTLD- | | | | | (26) |
| TDP | | | | | |
| MCF | 93 | 73.5 ± 10.7 | 44 (47.3%) | 43.0% | FTLD-TDP |
| FTLD- | | | | | (93) |
| TDP | | | | | |
| MC | 374 | 62.0 ± 10.5 | 188 (50.3%) | 45.7% | bvFTD (209), |
| clinical | | | | | FTD/ALS (16), |
| FTD | | | | | PNFA (76), SD |
| | | | | | (73) |
| MCF | 229 | 59.0 ± 11.3 | 104 (44.4%) | 14.8% | ALS (172), |
| clinical | | | | | ALS/FTD (31), |
| ALS | | | | | PMA (14), |
| | | | | | PMA/FTD (1), |
| | | | | | PLS (8), |
| | | | | | PLS/FTD (2), |
| | | | | | MMA(1)^{c} |
| Control | 909 | 75.0 ± 10.7 | 552 (60.7%) | n/a | n/a |

^{a}Age is shown as the median ± standard deviation, describing the age at onset for the clinical series, age at death for the pathologically confirmed series, and age at blood draw (clinical samples) or death (brain bank samples) for controls.
^{b}Positive family history in the FTLD-TDP and clinical FTD series is defined as a first or second degree relative with FTD and/or ALS or a first degree relative with memory problems, behavioral changes, Parkinsonism, schizophrenia, or another suspected neurodegenerative disorder. A positive family history in the clinical ALS series is defined as a first or second degree relative with ALS.
^{c}The MCF MMA patient had a family history of ALS.
ALS = amyotrophic lateral sclerosis; bvFTD = behavioral variant FTD; FTD = frontotemporal dementia; FTLD-TDP = Frontotemporal lobar degeneration with TDP-43 pathology; MMA = monomelic amyotrophy; PLS = primary lateral sclerosis; PMA = progressive muscular atrophy; PNFA = progressive non-fluent aphasia; SD = semantic dementia.

### Characterization of Hexanucleotide Repeat Insertion in C9ORF72 Genomic Region

The GGGGCC hexanucleotide repeat in C9ORF72 was PCR amplified in family VSM-20 and in all patient and control cohorts using the genotyping primers listed in Table 2 using one fluorescently labeled primer followed by fragment length analysis on an automated ABI3730 DNA-analyzer (Applied Biosystems). The PCR reaction was carried out in a mixture containing 1M betaine solution, 5% dimethylsulfoxide and 7-deaza-2-deoxy GTP in substitution for dGTP. Allele identification and scoring was performed using GeneMapper v4.0 software (Applied Biosystems). To determine the number of GGGGCC units and internal composition of the repeat, 48 individuals homozygous for different fragment lengths were sequenced using the PCR primers.

TABLE 2
Primer sequences.

| Technique | Primer name | Sequence |
|---|---|---|
| Genotyping | chr9:27563580F | FAM-CAAGGAGGGAAACAACCGCAGCC |
| | | (SEQ ID NO: 2) |
| | chr9:27563465R | GCAGGCACCGCAACCGCAG |
| | | (SEQ ID NO: 3) |
| Repeat primed PCR | MRX-F | FAM-TGTAAAACGACGGCCAGTCAAGGAGGG- |
| | | AAACAACCGCAGCC |
| | | (SEQ ID NO: 4) |
| | MRX-M13R | CAGGAAACAGCTATGACC |
| | | (SEQ ID NO: 5) |
| | MRX-R1 | CAGGAAACAGCTATGACCGGGCCCGCCCCGACC |
| | | ACGCCCCGGCCCCGGCCCCGG |
| | | (SEQ ID NO: 6) |
| Southern Blot | ProbeAF | AGAACAGGACAAGTTGCC |
| probe | | (SEQ ID NO: 7) |
| | ProbeAR | AACACACACCTCCTAAACC |
| | | (SEQ ID NO: 8) |
| rs3844942 SNP | Forward primer | CCCACAGGTCTAGCTAGTACGTAT |
| | | (SEQ ID NO: 9) |
| custom assay | Reverse primer | GACAAGAATCTTGTTCTTTAGCCTAGGT |
| | | (SEQ ID NO: 10) |
| | Reporter 1 | VIC-TGTAATAAATGCAATAAAAGAA |
| | | (SEQ ID NO: 11) |
| | Reporter 2 | FAM-AAATGCAACAAAAGAA |
| | | (SEQ ID NO: 12) |

### Repeat-Primed PCR Analysis

To provide a qualitative assessment of the presence of an expanded (GGGGCC)ₙ hexanucleotide repeat in C9ORF72, a repeat-primed PCR reaction was performed in the presence of 1M betaine, 5% dimethyl sulfoxide and complete substitution of 7-deaza-2-deoxy GTP for dGTP using a previously optimized and described cycling program (Hantash et al., Genet. Med., 12:162-173 (2010)). Primer sequences are set forth in Table 2. PCR products were analyzed on an ABI3730 DNA Analyzer and visualized using GeneMapper software.

### Probe Labeling, Agarose Gel Electrophoresis, Southern Transfer, Hybridization and Detection

A 241 bp digoxigenin (DIG)-labeled probe was generated using primers listed in Table 2 from 10 ng gDNA by PCR reaction using PCR DIG Probe Synthesis Kit Expand High fidelity mix enzyme and incorporating 0.35 mM DIG-11-dUTP: 0.65 mM dTTP (1:6) in the dNTP labeling mix as recommended in the DIG System User's Guide (Roche Applied Science). A total of 2 μL of PCR labeled probe per mL of hybridization solution was used as recommended in the DIG System User's Guide. A total of 5-10 μg of gDNA was digested with XbaI at 37° C. overnight and electrophoresed in 0.8% agarose gels in 1×TBE. DNA was transferred to positively charged nylon membrane (Roche Applied Science) by capillary blotting and crosslinked by UV irradiation. Following prehybridization in 20 mL DIG EasyHyb solution at 47° C. for 3 hours, hybridization was carried out at 47° C. overnight in a shaking water bath. The membranes were then washed two times in 2× standard sodium citrate (SSC), 0.1% sodium dodecyl sulfate (SDS) at room temperature for 5 minutes each and twice in 0.1×SSC, 0.1% SDS at 68° C. for 15 minutes each. Detection of the hybridized probe DNA was carried out as described in the User's Guide. CDP-star chemiluminescent substrate was used, and signals were visualized on X-ray film after 5 to 15 hours.

### SNP Genotyping

SNP rs3844942 was genotyped using a custom-designed Taqman SNP genotyping assay on the 7900HT Fast Real Time PCR system. Primers are set forth in Table 2. Genotype calls were made using the SDS v2.2 software (Applied Biosystems, Foster City, Calif.).

### C9ORF72 Quantitative Real-Time PCR

Total RNA was extracted from lymphoblast cell lines and brain tissue samples with the RNAeasy Plus Mini Kit (Qiagen) and reverse transcribed to cDNA using Oligo dT primers and the SuperScript III Kit (Invitrogen). RNA integrity was checked on an Agilent 2100 Bioanalyzer. Following standard protocols, real-time PCR was performed with inventoried TaqMan gene expression assays for GAPDH (Hs00266705) and C9ORF72 (Hs00945132) and one custom-designed assay specific to the C9ORF72 variant 1 transcript (Table 3) (Applied Biosystems) and analyzed on an ABI Prism 7900 system (Applied Biosystems). All samples were run in triplicate. Relative Quantification was determined using the ΔΔCₜ method after normalization to GAPDH. For the custom designed C9ORF72 variant 1 Taqman assay, probe efficiency was determined by generation of a standard curve (slope: −3.31459, r²: 0.999145).

TABLE 3
Custom TaqMan V1 specific assay sequences and gDNA/cDNA sequencing
primers.

| Technique | Primer name | Sequence |
|---|---|---|
| qPCR: custom assay | V1assay primer | CGGTGGCGAGTGGATATCTC |
| | F | (SEQ ID NO: 13) |
| | V1assay primer | TGGGCAAAGAGTCGACATCA |
| | R | (SEQ ID NO: 14) |
| | V1assay probe | TAATGTGACAGTTGGAATGC |
| | | (SEQ ID NO: 15) |
| gDNA sequencing | c9orf72-2aF | GGAGATAACAGGATTCCACATCTTTG |
| | | (SEQ ID NO: 16) |
| | c9orf72-2aR | CCACTCTCTGCATTTCGAAGGAT |
| | | (SEQ ID NO: 17) |
| cDNA sequencing & RT-PCR | cDNA V1 1F | CGGTGGCGAGTGGATATC |
| | | (SEQ ID NO: 18) |
| | cDNA V2 1F | AAGATGACGCTTGATATC |
| | | (SEQ ID NO: 19) |
| | cDNA V3 1F | GTGTGGGTTTAGGAGATATC |
| | | (SEQ ID NO: 20) |
| | cDNA 2F | CCGGAAAGGAAGAATATGG |
| | | (SEQ ID NO: 21) |
| | cDNA 2R | TATGAAGTGGGAGGTAGAAAC |
| | | (SEQ ID NO: 22) |
| | cDNA 5R | TTGAGAAGAAAGCCTTCATG |
| | | (SEQ ID NO: 23) |
| | cDNA 7F | AATATGAGTCAGGGCTCTTTGTAC |
| | | (SEQ ID NO: 24) |
| | cDNA 8R | TCGGATCTCATGTATCTACGC |
| | | (SEQ ID NO: 25) |
| | cDNA 11R | CCCTCTGCTGTTAAATCAAG |
| | | (SEQ ID NO: 26) |
| | β-actinF | GACAACGGCTCCGGCATGTG |
| | | (SEQ ID NO: 27) |
| | β-actinR | CCTTCTGACCCATGCCCAC |
| | | (SEQ ID NO: 28) |

C9ORF72 gDNA and cDNA Sequencing

To determine the genotype for rs10757668 in gDNA, C9ORF72 exon 2 was amplified using flanking primers c9orf72-2aF and c9orf72-2aR (Table 3). PCR products were purified using AMPure (Agencourt Biosciences) then sequenced in both directions with the same primers using the Big Dye Terminator v3.1 Cycle Sequencing kit (Applied Biosystems). Sequencing reactions were purified using CleanSEQ (Agencourt Biosciences) and analyzed on an ABI3730 Genetic Analyzer (Applied Biosystems). Sequence data was analyzed with Sequencher 4.5 software (Gene Codes). For cDNA sequencing, total RNA was isolated from frontal cortex tissue using the RNAeasy Plus Mini Kit (Qiagen). Reverse transcription reactions were performed using SuperScript III Kit (Invitrogen). RT-PCR was performed using primers specific for each of the three C9ORF72 mRNA transcripts; V1: cDNA-V1-1F with cDNA-2F, V2: cDNA-V2-1F with cDNA-2F, V3: cDNA-V3-1F with cDNA-2F (Table 2). PCR products were sequenced as described, and sequence data from each of the three transcripts were visualized for the genotype status of rs10757668.

C9orf72 Westernblot Analysis

Human-derived lymphoblast cells and frontal cortex tissue were homogenized in radioimmunoprecipitation assay (RIPA) buffer and protein content was measured by the BCA assay (Pierce). Twenty and fifty micrograms of protein were loaded for the lymphoblast and brain tissue lysates, respectively, and run on 10% SDS gels. Proteins were transferred onto Immobilon membranes (Invitrogen) and probed with antibodies against C9orf72 (Santa Cruz 1:5000 and GeneTex 1:2000). A GAPDH antibody (Meridian Life Sciences 1:500,000) was used as an internal control to verify equal protein loading between samples.

### RNA-FISH

For in situ hybridization, two 2′-O-methyl RNA 5′oligos labeled with Cy3 were ordered from IDT (Coralville, Iowa): (GGCCCC)₄ predicted to hybridize to the expanded GGGGCC repeat identified in this study and (CAGG)₆ predicted to hybridize only to CCTG repeats observed in DM2 and included in this experiment as a negative control. Slides were pre-treated following the in situ hybridization protocol from AbCam with minor modifications. Lyophilized probe was re-constituted to 100 ng/μL in nuclease free water. Probe working solutions of 5 ng/μL were used for paraffin specimens, and diluted in LSI/WCP Hybridization Buffer (Abbott Molecular). Following overnight hybridization, slides were washed 3 times in 1×PBS at 37° C. for 5 minutes each. DAPI counterstain (VectaShield®) was applied to each specimen and coverslipped. For each patient, 100 cells were scored for the presence of nuclear RNA foci per tissue section.

### Immunohistochemistry

Immunohistochemistry for C9ORF72 was performed on sections of post-mortem brain and spinal cord tissue from patients with FTLD-TDP pathology known to carry the GGGGCC repeat expansion (N=4), patients with FTLD-TDP without the repeat expansion (N=4), ALS without the repeat expansion (N=4), other molecular subtypes of FTLD (N=4), Alzheimer's disease (N=2) and neurologically normal controls (N=4). Immunohistochemistry was performed on 3 μm thick sections of formalin fixed, paraffin embedded post mortem brain and spinal cord tissue using the Ventana BenchMark® XT automated staining system (Ventana, Tucson, Ariz.) with anti-C9ORF72 primary antibody (Sigma-Aldrich, anti-C9orf72; 1:50 overnight incubation following microwave antigen retrieval) and developed with aminoethylcarbizole (AEC).

### Results

Expanded GGGGCC Hexanucleotide Repeat in C9ORF72 is the Cause of Chromosome 9p21-Linked FTD/ALS in Family VSM-20

In the process of sequencing the non-coding region of C9ORF72, a polymorphic GGGGCC hexanucleotide repeat (g.26724GGGGCC(3_{—}23) in the reverse complement of AL451123.12 starting at nt 1) located between non-coding C9ORF72 exons 1a and 1b was detected. Fluorescent fragment-length analysis of this region in samples from members of family VSM-20 resulted in an aberrant segregation pattern. All affected individuals appeared homozygous in this assay, and affected children appeared not to inherit an allele from the affected parent (FIG. 1A-B). To determine whether the lack of segregation was the result of single allele amplification due to the presence of an unamplifiable repeat expansion, a repeat-primed PCR method specifically designed to the observed GGGGCC hexanucleotide repeat was used. This method suggested the presence of repeat expansions in all affected members of family VSM-20, but not in unaffected relatives (FIG. 1C). Subsequent analysis of 909 healthy controls by fluorescent fragment-length analysis identified 315 who were homozygous, however no repeat expansions were observed by repeat-primed PCR. The maximum size of the repeat in controls was 23 units. These findings suggested the presence of a unique repeat expansion in family VSM-20. Southern blot analysis was perform on DNA from four different affected and one unaffected member of VSM-20. In addition to the expected normal allele, a variably sized expanded allele, too large to be amplified by PCR, which was found only in the affected individuals (FIG. 1D), was detected. In all but one patient, the expanded alleles appeared as single discrete bands; however, in patient 20-17 (FIG. 1D, lane 5) two discrete high molecular weight bands were observed, suggesting somatic instability of the repeat. Based on this small number of patients, it was estimated that the number of GGGGCC repeat units ranged from about 700 to 1600.

### Expanded GGGGCC Hexanucleotide Repeat in C9ORF72 is a Frequent Cause of Disease in FTD and ALS Patient Populations

The proband of family VSM-20 (20-6) was part of a highly selected series of 26 probands ascertained at UBC, Vancouver, Canada, with a confirmed pathological diagnosis of FTLD-TDP and a positive family history of FTD and/or ALS.

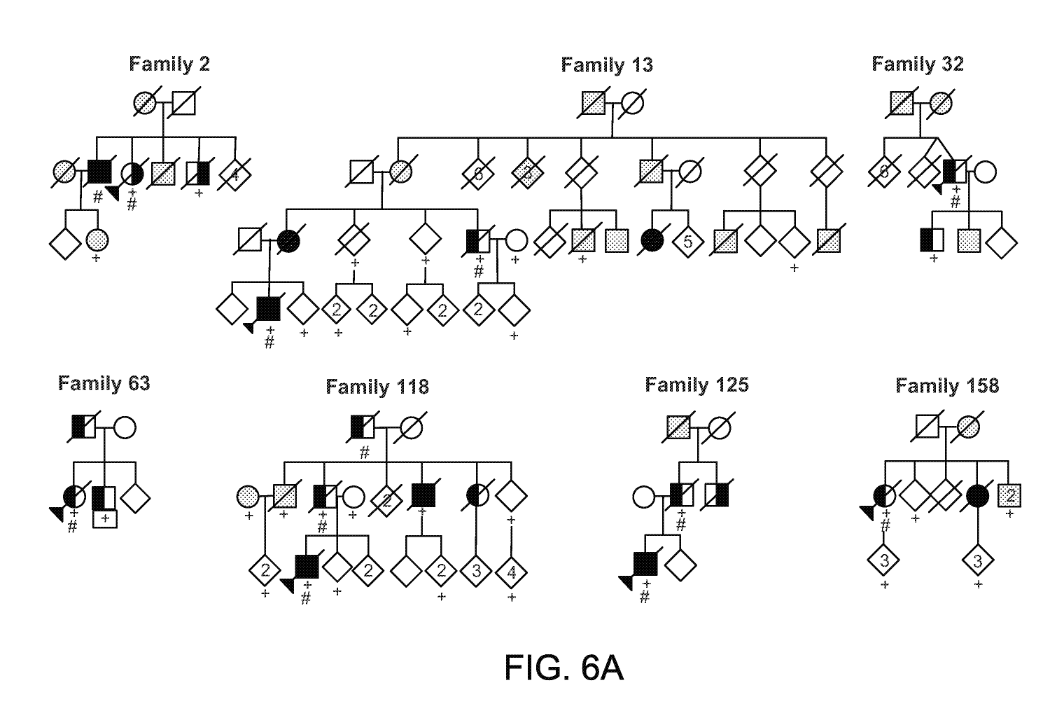

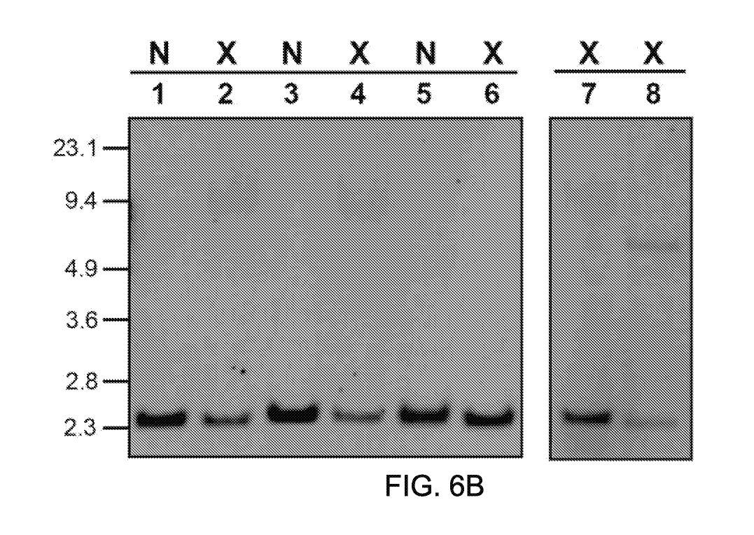

Using a combination of fluorescent fragment-length and repeat-primed PCR analyses, 16 of the 26 FTLD-TDP families in this series (61.5%) were found to carry expanded alleles of the GGGGCC hexanucleotide repeat; nine with a combined FTD/ALS phenotype and seven with clinically pure FTD. In five of these families, DNA was available from multiple affected members and in all cases, the repeat expansion was found to segregate with disease (FIG. 1 and FIG. 6). These findings suggest that GGGGCC expansions in C9ORF72 are the most common cause of familial FTLD-TDP.

To further determine the frequency of GGGGCC hexanucleotide expansions in C9ORF72 in patients with FTLD-TDP pathology and to assess the importance of this genetic defect in the etiology of patients clinically diagnosed with FTD and ALS, 696 patients (93 pathologically diagnosed FTLD-TDP, 374 clinical FTD, and 229 clinical ALS) derived from three well-characterized patient series ascertained at the Mayo Clinic Florida (MCF) and MCR were analyzed (Table 1). This resulted in the identification of 59 additional unrelated patients carrying GGGGCC repeat expansions, including 22 patients without a known family history (Table 4, FIG. 6). In a subset of these patients the sporadic nature of the disease could potentially be explained by the early death of one or both parents (3/22), adoption (1/22), or a lack of sufficient information (8/22); however, in 10 patients the clinical records suggested a true sporadic nature of the disease. The GGGGCC repeat was found in 18.3% of all patients with FTLD-TDP pathology from the MCF brain bank, and explained 22.5% of familial cases in this series. It should be noted however, that this is a dementia-focused series with an under-representation of ALS. The frequency in this clinical FTD patient series was 3.0% of sporadic cases and 11.7% of familial patients. In this clinical ALS series, 4.1% of the sporadic and 23.5% of patients with a positive family history carried repeat expansions. Importantly, a direct comparison of the frequency of repeat expansions in C9ORF72 with mutations in SOD1, TARDBP and FUS revealed GGGGCC expansions to be the most common genetic cause of sporadic and familial ALS in this clinical series (Table 4). In clinical FTD, GGGGCC repeat expansions were found to be more common than either GRN or microtubule associated protein tau (MAPT) mutations in familial cases, and of equal frequency to GRN mutations in sporadic FTD.

TABLE 4
Frequency of chromosome 9p repeat expansion in FTLD and ALS.

| | | Number of mutation carriers (%) | | | | | |
|---|---|---|---|---|---|---|---|
| Cohort | N | c9FTD/ALS | GRN | MAPT | SOD1 | TARDBP | FUS |
| UBC FTLD-TDP | | | | | | | |
| Familial | 26 | 16 (61.5) | 7 (26.9) | n/a | n/a | n/a | n/a |
| MCF FTLD-TDP | | | | | | | |
| Familial | 40 | 9 (22.5) | 6 (15.0) | n/a | n/a | n/a | n/a |
| Sporadic^{a} | 53 | 8 (15.1) | 8 (15.1) | n/a | n/a | n/a | n/a |
| MC Clinical FTLD | | | | | | | |
| Familial | 171 | 20 (11.7) | 13 (7.6) | 12 (6.3) | n/a | n/a | n/a |
| Sporadic | 203 | 6 (3.0) | 6 (3.0) | 3 (1.5) | n/a | n/a | n/a |
| MCF clinical ALS | | | | | | | |
| Familial | 34 | 8 (23.5) | n/a | n/a | 4 (11.8) | 1 (2.9) | 1 (2.9) |
| Sporadic | 195 | 8 (4.1) | n/a | n/a | 0 (0.0) | 2 (1.0) | 3 (1.5) |

^{a}Includes 22 individuals for which no information on family history was available.
UBC = University of British Columbia; MCF = Mayo Clinic Florida; MCM = Mayo Clinic Minnesota; FTLD-TDP = Frontotemporal lobar degeneration with TDP-43 pathology; ALS = Amyotrophic lateral sclerosis; c9FTD/ALS = (GGGGCC)ₙ repeat expansion at chromosome 9p identified in this study; GRN = Progranulin gene; MAPT = Microtubule associated protein tau gene; SOD1 = superoxide dismutase 1 gene; TARDBP = TAR DNA-binding protein 43 gene; FUS = fused in sarcoma gene; n/a = not applicable.

### Clinical and Pathological Characteristics of Expanded GGGGCC Repeat Carriers

Clinical data was obtained for the 26 unrelated expanded repeat carriers from the clinical FTD series and the 16 unrelated carriers from the ALS series. The median age of onset was comparable in the two series (FTD: 56.2 years, range 34-72 years; ALS: 54.5 years, range 41-72 years), with a slightly shorter mean disease duration in the ALS patients (FTD: 5.1±3.1 years, range 1-12 years, N=18; ALS: 3.6±1.6 years, range 1-6 years, N=7). The FTD phenotype was predominantly behavioral variant FTD (bvFTD) (25/26). Seven patients from the FTD series (26.9%) had concomitant ALS, and eight patients (30.7%) had relatives affected with ALS. In comparison, the frequency of a family history of ALS in the remainder of the FTD population (those without repeat expansions) was only 5/348 (1.4%). In the ALS series, all mutation carriers presented with classical ALS with the exception of one patient diagnosed with progressive muscular atrophy without upper motor neuron signs. Three patients (18.8%) were diagnosed with a combined ALS/FTD phenotype. In the ALS patients with expanded repeats, 11/16 (68.8%) reported relatives with FTD or dementia, compared to only 61/213 (28.6%) of ALS patients without repeat expansions. Finally, autopsy was subsequently performed on 11 FTD and three ALS expanded repeat carriers from the clinical series, and in all cases, TDP-43 based pathology was confirmed.

Comparison of Haplotypes Carrying Expanded GGGGCC Repeats with Previously Reported Chromosome 9p ‘Risk’ Haplotype

A ˜140 kb risk haplotype on chromosome 9p21 was shared by four chromosome 9p-linked families and exhibited significant association with FTD and ALS in at least eight populations. To determine whether all GGGGCC expanded repeat carriers identified herein also carried this ‘risk’ haplotype, and to further study the significance of this finding, the variant rs3849942 was selected as a surrogate marker for the ‘risk’ haplotype for genotyping in these patient and control populations. All 75 unrelated expanded repeat carriers had at least one copy of the ‘risk’ haplotype (100%) compared to only 23.1% of the control population. In order to associate the repeat sizes with the presence or absence of the ‘risk’ haplotype, we further focused on controls homozygous for rs3849942 (505 GG and 49 AA) and determined the distribution of the repeat sizes in both groups (FIG. 2). A striking difference was found in the number of GGGGCC repeats, with significantly longer repeats on the ‘risk’ haplotype tagged by allele ‘A’ compared to the wild-type haplotype tagged by allele ‘G’ (median repeat length: risk haplotype=8, wild-type haplotype=2; average repeat length: risk haplotype=9.5, wild-type haplotype=3.0; p<0.0001). Sequencing analysis of 48 controls in which the repeat length was the same on both alleles (range=2-13 repeat units) further showed that the GGGGCC repeat was uninterrupted in all individuals.

### Expanded GGGGCC Repeat Affects C9ORF72 Expression in a Transcript-Specific Manner

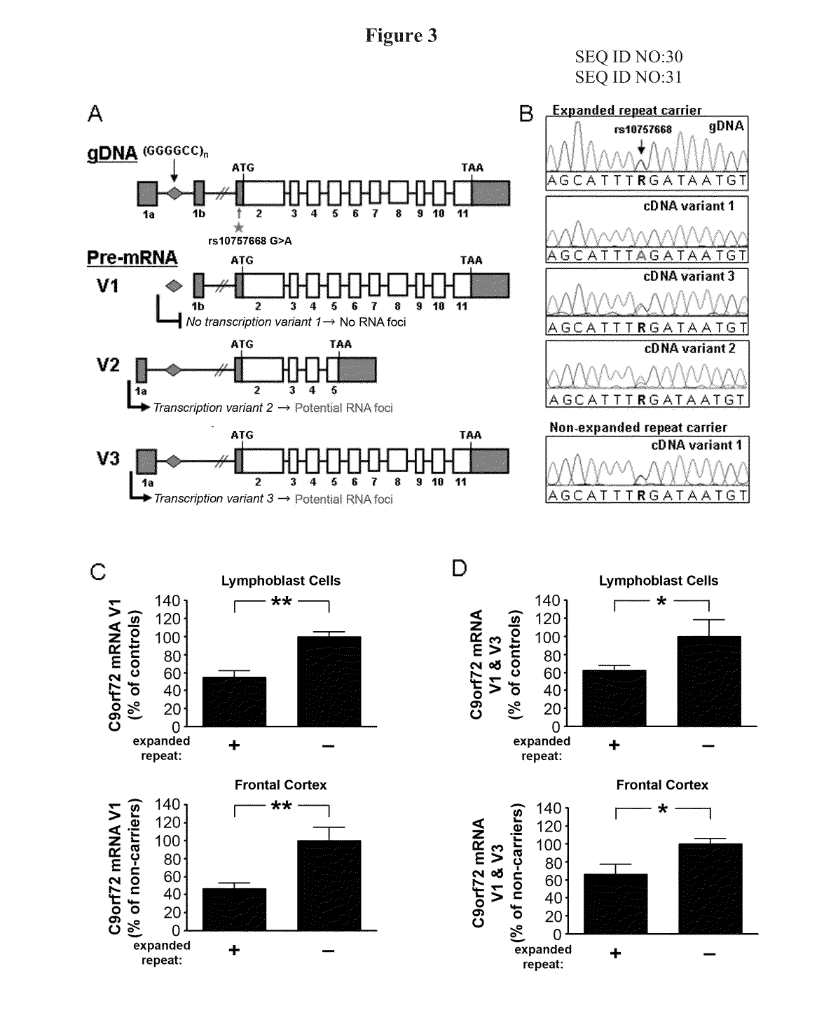

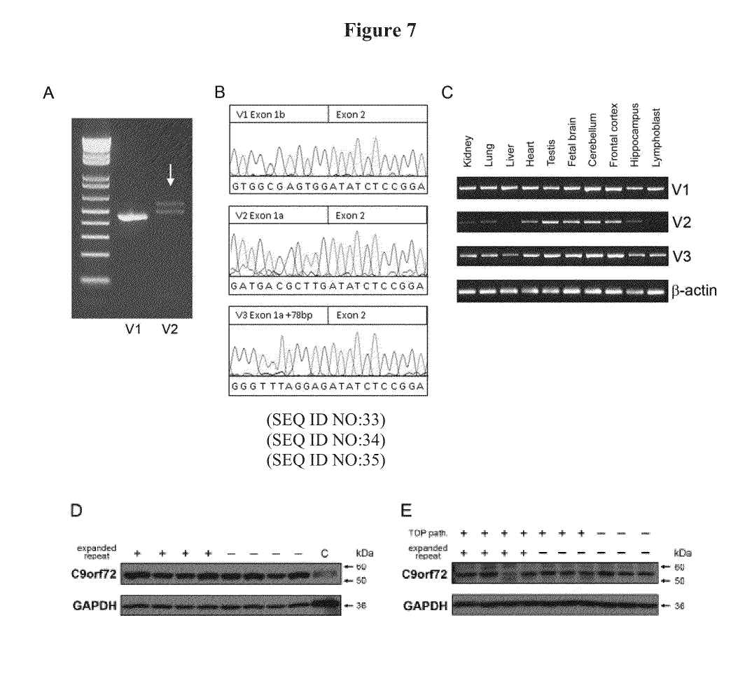

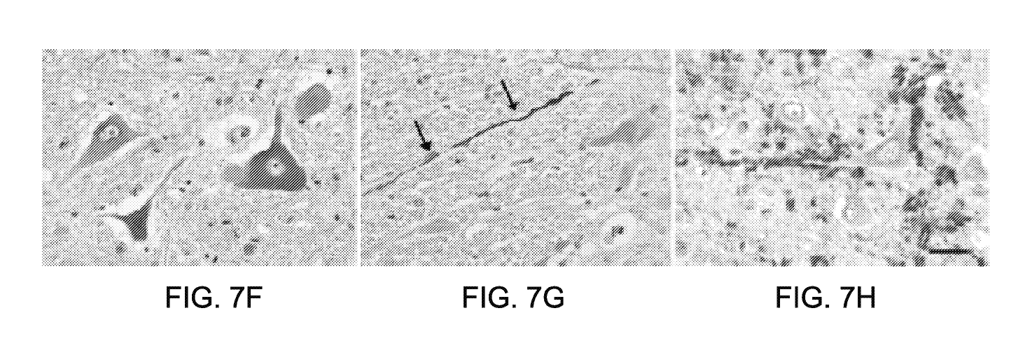

One mechanism by which expansion of a non-coding repeat region might lead to disease is by interfering with normal expression of the encoded protein. Through a complex process of alternative splicing, three C9ORF72 transcripts were produced which were predicted to lead to the expression of two alternative isoforms of the uncharacterized protein C9ORF72 (FIG. 3A). Transcript variants 1 and 3 were predicted to encode for a 481 amino acid long protein encoded by C9ORF72 exons 2-11 (NP_{—}060795.1; isoform a), whereas variant 2 was predicted to encode a shorter 222 amino acid protein encoded by exons 2-5 (NP_{—}659442.2; isoform b) (FIG. 3A). RT-PCR analysis showed that all C9ORF72 transcripts were present in a variety of tissues, and immunohistochemical analysis in brain further showed that C9ORF72 was largely a cytoplasmic protein in neurons (FIG. 7).

The GGGGCC hexanucleotide repeat was located between two alternatively-spliced non-coding first exons, and depending on their use, the expanded repeat was either located in the promoter region (for transcript variant 1) or in intron 1 (for transcript variants 2 and 3) of C9ORF72 (FIG. 3A). This complexity raised the possibility that the expanded repeat affects C9ORF72 expression in a transcript-specific manner. To address this, we first determined whether each of the three C9ORF72 transcripts, carrying the expanded repeat, produce mRNA expression in brain. For this, two GGGGCC repeat carriers were selected for which frozen frontal cortex brain tissue was available and who were heterozygous for the rare sequence variant rs10757668 in C9ORF72 exon 2. Comparison of sequence traces of C9ORF72 exon 2 in gDNA and transcript-specific cDNAs amplified from these patients revealed the absence of variant 1 transcribed from the mutant RNA (G-allele) but normal transcription of variant 2 and 3 (FIG. 3B). The loss of variant 1 expression in the GGGGCC repeat carriers was further confirmed by real-time RT-PCR using a custom-designed Taqman assay specific to variant 1.

In lymphoblast cell lines of patients from family VSM-20 and in frontal cortex samples from unrelated FTLD-TDP patients carrying expanded repeats, the level of C9ORF72 variant 1 was approximately 50% reduced compared to non-repeat carriers (FIG. 3C). Since C9ORF72 variants 1 and 3, which each contain a different non-coding first exon, both encode C9ORF72 isoform a (NP_{—}060795.1), we next determined the effect of the expanded repeats on the total levels of transcripts encoding this isoform (variants 1 and 3 combined) using an inventoried ABI Taqman assay (Hs_{—}00945132). Significant mRNA reductions were observed in both lymphoblast cells (34% reduction) and frontal cortex samples (38% reduction) from expanded repeat carriers (FIG. 3D). In contrast, no appreciable changes in total levels of C9ORF72 protein could be observed by western blot analysis of lymphoblast cell lysates or brain (FIG. 7) or by immunohistochemical analysis of C9ORF72 in post-mortem brain or spinal cord tissue from expanded repeat carriers (FIG. 7).

### The Transcribed GGGGCC Repeat Forms Nuclear RNA Foci in Affected Central Nervous System Regions of Mutation Carriers

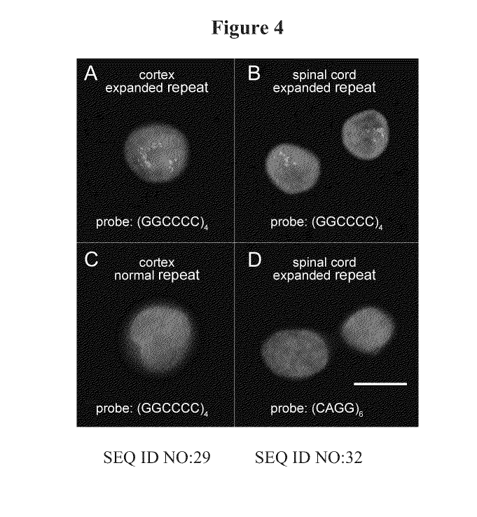

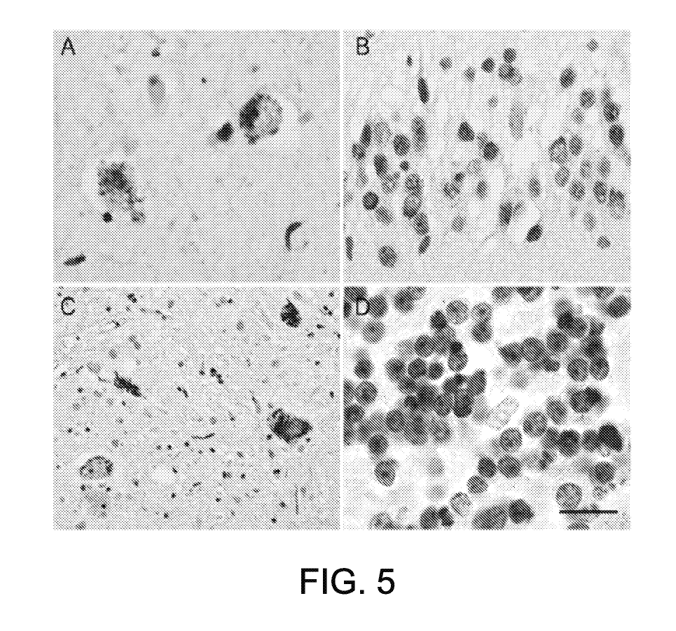

A second mechanism by which abnormal expansion of a non-coding repeat region can cause neurological disease is through the intracellular accumulation of the nucleotide repeat as RNA foci (Todd and Paulson, Ann. Neurol., 67:291-300 (2010)). To determine whether the GGGGCC repeat in C9ORF72 results in RNA foci, RNA fluorescence in situ hybridization (FISH) in paraffin-embedded sections of post-mortem frontal cortex and spinal cord tissue from FTLD-TDP patients was performed. For each neuroanatomical region, sections from two patients with expanded GGGGCC repeats and two affected patients with normal repeat lengths were analyzed. Using a probe targeting the GGGGCC repeat (probe (GGCCCC)₄), multiple RNA foci were detected in the nuclei of 25% of cells in both the frontal cortex and the spinal cord from patients carrying the expansion, whereas a signal was observed in only 1% of cells in tissue sections from non-carriers (FIG. 4A-C). Foci were never observed in any of the samples using a probe targeting the unrelated CCTG repeat (probe (CAGG)₆), implicated in myotonic dystrophy type 2 (DM2) (Liquori et al., Science, 293:864-867 (2001)), further supporting the specificity of the RNA foci composed of GGGGCC in these patients (FIG. 4D).

Taken together, these results demonstrate the identification of a non-coding expanded GGGGCC hexanucleotide repeat in C9ORF72 as the cause of chromosome 9p-linked FTD/ALS and demonstrate that this genetic defect is a common cause of ALS and FTD identified. There results also demonstrate multiple potential disease mechanisms associated with this repeat expansion, including a direct effect on C9ORF72 expression by affecting transcription (loss-of-function mechanism) and an RNA-mediated gain-of-function mechanism through the generation of toxic RNA foci.

### Example 2

### Somatic Heterogeneity of the GGGGCC Hexanucleotide Repeat in C9ORF72 Expanded Repeat Carriers

The following was performed to determine the GGGGCC repeat size and degree of heterogeneity in DNA samples from different brain regions and non-affected peripheral tissues in C9ORF72 mutation carriers. Three ALS patients with C9ORF72 expanded repeats ascertained at the ALS Center at Mayo Clinic Florida with full autopsy available at the Mayo Clinic Florida Brain Bank were studied. Genomic DNA (gDNA) was extracted from blood, spleen, heart, muscle, liver, and different brain regions (frontal cortex, temporal cortex, parietal cortex, occipital cortex and cerebellum) and used for southern blot analysis.

The C9ORF72 mutation carriers all presented clinical features of classical ALS with the exception of one patient diagnosed with progressive muscular atrophy (PMA) without upper motor neuron signs. TDP-43-positive pathology was confirmed in all patients. Post-mortem examination revealed classical ALS pathology in two cases and FTLD-MND with predominantly lower motor pathology in the PMA patient.

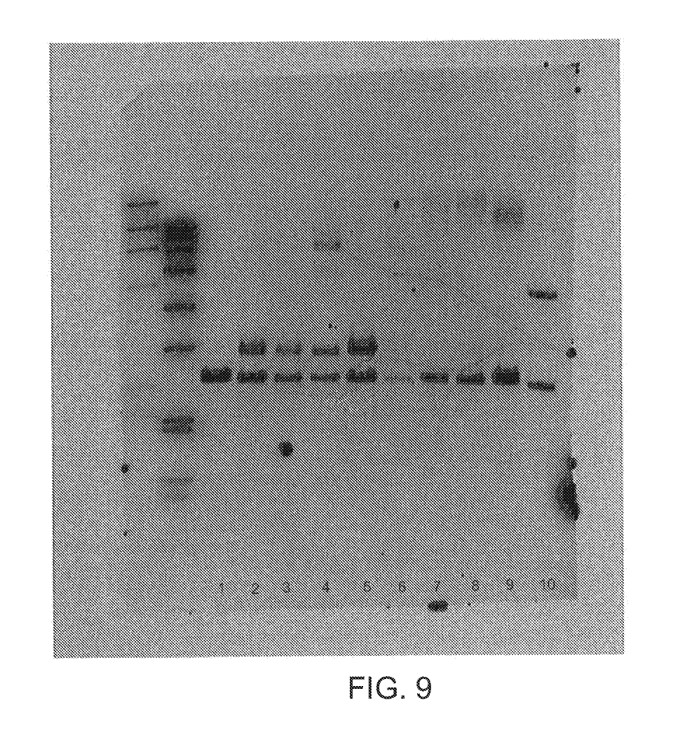

Southern blot analysis using DNA extracted from several brain regions, peripheral tissues, and blood confirmed the presence of an expanded allele with a smear of high molecular weight bands in all cases, suggesting somatic instability of the expanded repeat (see, e.g., FIG. 9). Direct repeat size comparison of gDNA from blood and cerebellum revealed no significant difference in size in two cases, whereas the third case diagnosed with PMA exhibited only 80-100 repeats in blood and >1000 repeats in the cerebellum (FIG. 9).

Variable degrees of somatic heterogeneity of repeat size in the expanded alleles within and across tissues in all affected individuals were detected. The longest repeat lengths were generally observed in brain. These results demonstrate that the repeat length in C9ORF72 mutation carriers is highly variable across tissues as a result of somatic instability.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for diagnosing frontotemporal dementia or amyotrophic lateral sclerosis, wherein said method comprises:
(a) detecting the presence of an expanded number of GGGGCC repeats located in a C9ORF72 nucleic acid of a human, and
(b) classifying said human as having frontotemporal dementia or amyotrophic lateral sclerosis based at least in part on the detection of said presence.

2. The method of claim 1, wherein said GGGGCC repeats are located in a non-coding region of said C9ORF72 nucleic acid.

3. The method of claim 1, wherein said method comprises detecting the presence of greater than 100 GGGGCC repeats.

4. The method of claim 1, wherein said method comprises detecting the presence of greater than 500 GGGGCC repeats.

5. The method of claim 1, wherein said detecting step comprises performing a polymerase chain reaction assay.

6. The method of claim 1, wherein said detecting step comprises performing a Southern blot assay.

7. An isolated nucleic acid comprising a C9ORF72 nucleic acid sequence having greater than 50 GGGGCC repeats.

8. An isolated nucleic acid comprising a C9ORF72 nucleic acid sequence having greater than 100 GGGGCC repeats.

9. An isolated nucleic acid molecule for performing a Southern blot analysis, wherein said isolated nucleic acid molecule comprises a C9ORF72 nucleic acid sequence having greater than 20 GGGGCC repeats.

10. A container comprising a population of isolated nucleic acid molecules, wherein said isolated nucleic acid molecules comprise a C9ORF72 nucleic acid sequence having greater than 10 GGGGCC repeats, wherein said population comprises at least five different isolated nucleic acid molecules each with a different number of GGGGCC repeats.

11. The container of claim 10, wherein said isolated nucleic acid molecules comprise a fluorescent label.


SEQUENCE LISTING (35 sequences)

SEQ ID NO: 1 - DNA, 6 bp, Homo sapiens
ggggcc

SEQ ID NO: 2 - DNA, 23 bp, Homo sapiens
caaggagggaaacaaccgcagcc

SEQ ID NO: 3 - DNA, 19 bp, Homo sapiens
gcaggcaccgcaaccgcag

SEQ ID NO: 4 - DNA, 41 bp, Homo sapiens
tgtaaaacgacggccagtcaaggagggaaacaaccgcagcc

SEQ ID NO: 5 - DNA, 18 bp, Artificial Sequence
  synthetically generated oligonucleotide
caggaaacagctatgacc

SEQ ID NO: 6 - DNA, 54 bp, Artificial Sequence
  synthetically generated oligonucleotide
caggaaacagctatgaccgggcccgccccgaccacgccccggccccggccccgg

SEQ ID NO: 7 - DNA, 18 bp, Homo sapiens
agaacaggacaagttgcc

SEQ ID NO: 8 - DNA, 19 bp, Homo sapiens
aacacacacctcctaaacc

SEQ ID NO: 9 - DNA, 24 bp, Homo sapiens
cccacaggtctagctagtacgtat

SEQ ID NO: 10 - DNA, 28 bp, Homo sapiens
gacaagaatcttgttctttagcctaggt

SEQ ID NO: 11 - DNA, 22 bp, Homo sapiens
tgtaataaatgcaataaaagaa

SEQ ID NO: 12 - DNA, 16 bp, Homo sapiens
aaatgcaacaaaagaa

SEQ ID NO: 13 - DNA, 20 bp, Homo sapiens
cggtggcgagtggatatctc

SEQ ID NO: 14 - DNA, 20 bp, Homo sapiens
tgggcaaagagtcgacatca

SEQ ID NO: 15 - DNA, 20 bp, Homo sapiens
taatgtgacagttggaatgc

SEQ ID NO: 16 - DNA, 26 bp, Homo sapiens
ggagataacaggattccacatctttg

SEQ ID NO: 17 - DNA, 23 bp, Homo sapiens
ccactctctgcatttcgaaggat

SEQ ID NO: 18 - DNA, 18 bp, Homo sapiens
cggtggcgagtggatatc

SEQ ID NO: 19 - DNA, 18 bp, Homo sapiens
aagatgacgcttgatatc

SEQ ID NO: 20 - DNA, 20 bp, Homo sapiens
gtgtgggtttaggagatatc

SEQ ID NO: 21 - DNA, 19 bp, Homo sapiens
ccggaaaggaagaatatgg

SEQ ID NO: 22 - DNA, 21 bp, Homo sapiens
tatgaagtgggaggtagaaac

SEQ ID NO: 23 - DNA, 20 bp, Homo sapiens
ttgagaagaaagccttcatg

SEQ ID NO: 24 - DNA, 24 bp, Homo sapiens
aatatgagtcagggctctttgtac

SEQ ID NO: 25 - DNA, 21 bp, Homo sapiens
tcggatctcatgtatctacgc

SEQ ID NO: 26 - DNA, 20 bp, Homo sapiens
ccctctgctgttaaatcaag

SEQ ID NO: 27 - DNA, 20 bp, Homo sapiens
gacaacggctccggcatgtg

SEQ ID NO: 28 - DNA, 19 bp, Homo sapiens
ccttctgacccatgcccac

SEQ ID NO: 29 - DNA, 24 bp, Homo sapiens
ggccccggccccggccccggcccc

SEQ ID NO: 30 - DNA, 16 bp, Homo sapiens
agcatttrgataatgt

SEQ ID NO: 31 - DNA, 16 bp, Homo sapiens
agcatttagataatgt

SEQ ID NO: 32 - DNA, 24 bp, Homo sapiens
caggcaggcaggcaggcaggcagg

SEQ ID NO: 33 - DNA, 22 bp, Homo sapiens
gtggcgagtggatatctccgga

SEQ ID NO: 34 - DNA, 22 bp, Homo sapiens
gatgacgcttgatatctccgga

SEQ ID NO: 35 - DNA, 22 bp, Homo sapiens
gggtttaggagatatctccgga